(19) United States
(12) Patent Application Publication
(10) Pub. No.: US 2015/0346586 A1
(43) Pub. Date: Dec. 3, 2015

(54) LIGHT EMITTING DIODE PACKAGING STRUCTURE AND CAMERA MODULE USING THE SAME

(71) Applicant: ALTEK CORPORATION, Hsinchu (TW)

(72) Inventor: Shu-Yu LIN, New Taipei City (TW)

(21) Appl. No.: 14/326,056

(22) Filed: Jul. 8, 2014

(30) Foreign Application Priority Data
May 30, 2014 (TW) 103118905

(51) Int. Cl. G03B 15/05; H01L 33/48; H04N 5/225; H01L 33/62; A61B 1/04; H01L 25/075; H01L 33/54

(52) U.S. Cl. CPC G03B 15/05; H01L 25/0753; H01L 33/486; H01L 33/54; H01L 33/62; A61B 1/04; H04N 5/2257; H04N 5/2256; H04N 5/2254; H01L 2933/0091

(57) ABSTRACT

The present disclosure illustrates a light emitting diode packaging structure and a camera module having the same. The light emitting diode packaging structure comprises a transparent package member, a plurality of light emitting diodes, and a plurality of conductive wires. The transparent package member has a geometrical shape with an opening located at the center thereof. The opening tightly fits with lens module. The plurality of light emitting diodes are located in the transparent package member. The plurality of light emitting diodes are electrically connected with the plurality of conductive wires, and parts of the plurality of conducting wires are exposed to outside of the transparent package for receiving electric power to provide driving power to the plurality of light emitting diodes. The camera module comprises an image sensor, a lens module, and the light emitting diode packaging structure.

### CROSS-REFERENCE TO RELATED APPLICATION

This application claims the benefit of Taiwan Patent Application No. 103118905, filed on May 30, 2014, in the Taiwan Intellectual Property Office, the disclosure of which is incorporated herein in its entirety by reference.

### BACKGROUND OF THE INVENTION

1. Field of the Invention

The present disclosure relates to a light emitting diode packaging structure, in particular, to a light emitting diode packaging structure adapted for a camera module of an endoscope.

2. Description of the Related Art

An endoscope is a long and flexible tube apparatus, and mainly includes an image capture device and a light source. After the endoscope is connected to a displayer, organs in human's body, inner sight of a vehicle, components within an electronic device and cracks of building structure can be imaging by the endoscope and shown on the displayer. The endoscope can be applied in application of industry and medical treatment. When the organ inside the body gets hurt and a tool component is damaged, the endoscope can be utilized to inspect so long as a channel exists between the organ or the damaged tool component and the outside. For example, an endoscope for upper digestive tract such as esophagus, stomach and duodenum, it is inserted through oral cavity. A colonoscope is inserted through the anus. An endoscope for a turbine blade is inserted through an exhaust port.

As miniaturization for the lens and the light emitting diode are developed maturely, current endoscope has the lens and the light source disposed at the front end thereof, and the front end including the lens and the light source is inserted inside the body. A cover glass is covered on an outer layer of the front end to prevent the lens or the light source from dropping off. However, based on light reflection principle, light emitted from the light source is possibly reflected by the cover glass and enters to the image sensor unit, which results in a white spot effect in the captured image.

Therefore, what is need is a camera module to solve the above-mentioned problems.

### SUMMARY OF THE INVENTION

An aspect of exemplary embodiments of the present disclosure directs to a light emitting diode packaging structure adapted for a camera module of endoscope, to avoid occurrence of white spot during image capture, to improve quality of captured image.

An exemplary embodiment of the present disclosure provides a light emitting diode packaging structure adapted for a lens module. The light emitting diode packaging structure includes a transparent package member, a plurality of light emitting diodes, and a plurality of conductive wires. The transparent package member is in geometrical shape including an opening which tightly fits with the lens module. The plurality of light emitting diodes are located in the transparent package member. The plurality of light emitting diodes are electrically connected with the plurality of conductive wires, and parts of the plurality of conducting wires are exposed to outside of the transparent package for receiving electric power to provide driving power to the plurality of light emitting diodes.

Preferably, the transparent package member further includes a plurality of light scattering particles which are disposed in a surface of the transparent package member to scatter the light emitted from the plurality of light emitting diodes.

Preferably, the plurality of light emitting diodes are distributed annularly inside the transparent package member.

Preferably, the plurality of conductive wires electrically connect a plurality of light emitting diodes with each other so as to form a series circuit.

Preferably, the transparent package member is adhesive to the lens module by UV glue.

An exemplary embodiment of the present disclosure provides a camera module includes an image sensor, a lens module, and a light emitting diode packaging structure. The lens module is disposed on the image sensor unit. The light emitting diode packaging structure includes a transparent package member, a plurality of light emitting diodes, and a plurality of conductive wires. The transparent package member has a geometrical shape with an opening located at the center thereof. The plurality of light emitting diodes are located in the transparent package member. The plurality of light emitting diodes are electrically connected with the plurality of conductive wires, and parts of the plurality of conducting wires are exposed to outside of the transparent package for receiving electric power to provide driving power to the plurality of light emitting diodes.

Preferably, the transparent package member further includes a plurality of light scattering particles which are disposed in a surface of the transparent package member to scatter the light emitted from the plurality of light emitting diodes.

Preferably, the plurality of light emitting diodes are distributed annularly inside the transparent package member, and each of the plurality of light emitting diodes includes a light emitting diode die.

Preferably, the plurality of conductive wires are connected electrically to a plurality of light emitting diodes to form a series circuit.

Preferably, the transparent package member is adhesive to the lens module by an UV glue.

### BRIEF DESCRIPTION OF THE DRAWINGS

The detailed structure, operating principle and effects of the present disclosure will now be described in more details hereinafter with reference to the accompanying drawings that show various embodiments of the present disclosure as follows.

FIG. 1 is a schematic view of an exemplary embodiment of a light emitting diode packaging structure according to the present disclosure.

FIG. 2 is a schematic view of an exemplary embodiment of a camera module of the present disclosure.

### DETAILED DESCRIPTION OF THE PREFERRED EMBODIMENTS

Reference will now be made in detail to the exemplary embodiments of the present disclosure, examples of which are illustrated in the accompanying drawings. Therefore, it is to be understood that the foregoing is illustrative of exemplary embodiments and is not to be construed as limited to the specific embodiments disclosed, and that modifications to the disclosed exemplary embodiments, as well as other exemplary embodiments, are intended to be included within the scope of the appended claims. These embodiments are provided so that this disclosure will be thorough and complete, and will fully convey the inventive concept to those skilled in the art. The relative proportions and ratios of elements in the drawings may be exaggerated or diminished in size for the sake of clarity and convenience in the drawings, and such arbitrary proportions are only illustrative and not limiting in any way. The same reference numbers are used in the drawings and the description to refer to the same or like parts.

It will be understood that, although the terms ‘first’, ‘second’, ‘third’, etc., may be used herein to describe various elements, these elements should not be limited by these terms. The terms are used only for the purpose of distinguishing one component from another component. Thus, a first element discussed below could be termed a second element without departing from the teachings of embodiments. As used herein, the term “or” includes any and all combinations of one or more of the associated listed items.

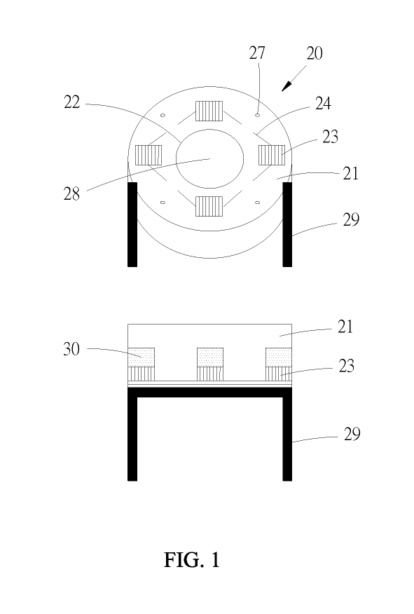

Please refer to FIG. 1 which shows a schematic view of an exemplary embodiment of a light emitting diode packaging structure of the present disclosure. The light emitting diode packaging structure 20 includes a transparent package member 21, a plurality of light emitting diodes 23 and a plurality of conductive wires 24. The material of the transparent package member 21 includes high transparent resin, such as epoxy resin or silicone.

The transparent package member 21 is in geometrical shape including an opening 22. The geometrical shape includes a circle shape or an elliptical shape. In implementation, the opening 22 includes a circle opening, when the transparent package member 21 is circle or elliptical shape, the light emitting diode packaging structure 20 is a ring type light emitting diode packaging structure. The opening 22 can tightly fit with a lens module (not shown in FIGs), and the transparent package member 21 and the lens module can be adhesive tightly by UV glue. The plurality of light emitting diodes 23 are located and distributed inside the transparent package member 21, such as in annular distribution. Apart from the light emitting diodes 23, the transparent package member 21 further includes a plurality of light scattering particles 27 which are disposed in a surface of the transparent package member 21 to scatter the light emitted from the plurality of light emitting diodes 23. The plurality of light emitting diodes 23 are electrically connected with the plurality of conductive wires 24, and parts of the plurality of conducting wires 24 are exposed to outside of the transparent package member 21 for receiving electric power to provide driving power to the plurality of light emitting diodes 23. The plurality of conductive wires 24 electrically connect the plurality of light emitting diodes 23 with each other so as to form a series circuit.

A support 29 can further be disposed under the transparent package member 21 which has an opening 22 and the plurality of light emitting diodes 23 distributed annularly. The plurality of light emitting diodes 23 include phosphor powder 30 which can be excited to emit different color light. A reserved space 28 in the opening 22 can be used to fit tightly with the lens module, and the plurality of light emitting diodes 23 are connected in series by the plurality of conductive wires 24.

In detail, the light emitting diode packaging structure 20 can form an annular light source, the surface of the annular light source can be processed to control the light scattered from the light emitting diode 23. The opening 22 can fit with the lens module directly. The light emitting diode packaging structure 20 can replace the traditional lens module of the light source circuit board directly. Moreover, the light emitting diode packaging structure 20 of present disclosure also can replace the traditional cover or cover glass used in the lens module of the endoscope, and the white spot problem caused by the cover glass can be prevented.

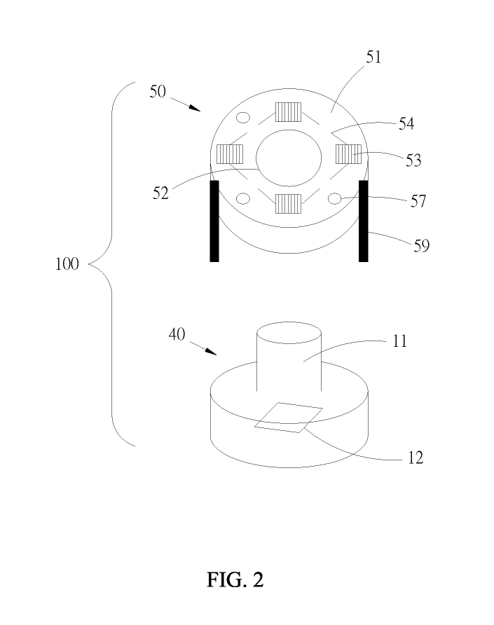

Please refer to FIG. 2 which is a schematic view of an exemplary embodiment of a camera module of the present disclosure. As shown in FIG. 2, the camera module 100 includes a lens module 40 and the light emitting diode packaging structure 50 of the above-mentioned exemplary embodiment. Particularly, the camera module 100 of the exemplary embodiment is adapted for an endoscope, and the lens module 40 can comprise a wafer-level lens module. For example, the lens module 40 includes a lens module 11 and an image sensor unit 12. The lens module 11 is disposed above the image sensor unit 12, and includes at least one lens. The light emitting diode packaging structure 50 includes a transparent package member 51, a plurality of light emitting diode 53, and a plurality of conductive wires 54. The light emitting diode packaging structure 50 is in a geometrical shape including an opening 52. The light emitting diode packaging structure 50 can be mounted on the lens module 40 via the opening 52. The plurality of light emitting diodes 53 are located in the transparent package member 51. The plurality of light emitting diodes 53 are electrically connected to the plurality of conductive wires 54, and parts of the plurality of conducting wires 54 are exposed to outside of the transparent package member 51 for receiving electric power to provide driving power to the plurality of light emitting diodes 53. In addition, the light emitting diode packaging structure 50 can be mounted on the lens module 40 by the support 59.

For example, the lens module 11 can include at least one lens (not shown in FIGs) and an infrared cut-off filter (not shown in FIGs), and the image sensor unit 26 can include a charge coupled device or CMOS image sensor unit.

For example, UV glue can be coated on the opening 52 of the light emitting diode packaging structure 50, to bind tightly the opening 52 and the lens module 40 for preventing from dropping off. In addition, the transparent package member 51 further includes a plurality of light scattering particles 57 which are located on a surface of the transparent package member 51 to scatter the light emitted from a plurality of light emitting diode 53, so that a reference light source with sufficient luminance can be provided while the lens module 11 captures image of outside.

In summary, the camera module of the present disclosure does not use the cover glass, so the white spot effect caused by the cover glass during image capturing, and the quality of captured image can be improved. In addition, extra components mounted on the lens and image sensor unit can be reduced by using the packaging process, so an efficient structure arrangement can be achieved, and the misalignment caused during traditional assembly of the lens and the image sensor on the endoscope can also be reduce for improving the yield rate.

The above-mentioned descriptions represent merely the exemplary embodiment of the present disclosure, without any intention to limit the scope of the present disclosure thereto. Various equivalent changes, alternations or modifications based on the claims of present disclosure are all consequently viewed as being embraced by the scope of the present disclosure.

What is claimed is:

1. A light emitting diode packaging structure, adapted for a lens module, the light emitting diode packaging structure comprising:
a transparent package member, being in a geometrical shape and having an opening which tightly fits with the lens module;
a plurality of light emitting diodes, located in the transparent package member; and
a plurality of conductive wires, connected with the plurality of light emitting diodes, respectively, parts of the plurality of conducting wires being exposed to outside of the transparent package member for receiving electric power to provide driving power to the plurality of light emitting diodes.

2. The light emitting diode packaging structure according to claim 1, wherein the transparent package member further comprises a plurality of light scattering particles which are disposed in a surface of the transparent package member to scatter the light emitted from the plurality of light emitting diode.

3. The light emitting diode packaging structure according to claim 1, wherein the plurality of light emitting diodes are distributed annularly inside the transparent package member.

4. The light emitting diode packaging structure according to claim 1, wherein the plurality of conductive wires electrically connect the plurality of light emitting diodes with each other so as to form a series circuit.

5. The light emitting diode packaging structure according to claim 1, wherein the transparent package member is adhesive to the lens module by UV glue.

6. A camera module, comprising:
a lens module, including an image sensor unit and a lens module which is disposed above the image sensor unit; and
a light emitting diode packaging structure, comprising:
a transparent package member, being in a geometrical shape having an opening which tightly fits with the lens module;
a plurality of light emitting diodes, located in the transparent package member; and
a plurality of conductive wires, connected with the plurality of light emitting diodes, respectively, parts of the plurality of conducting wires being exposed to outside of the transparent package member for receiving electric power to provide driving power to the plurality of light emitting diodes.

7. The camera module according to claim 6, wherein the transparent package member further comprises a plurality of light scattering particles which are disposed in a surface of the transparent package member to scatter the light emitted from the plurality of light emitting diode.

8. The camera module according to claim 6, wherein the plurality of light emitting diodes are distributed annularly inside the transparent package member.

9. The camera module according to claim 6, wherein the plurality of conductive wires electrically connect the plurality of light emitting diodes with each other so as to form a series circuit.

10. The camera module according to claim 6, wherein the transparent package member is adhesive to the lens module by UV glue.
